(19) United States
(12) Patent Application Publication
(10) Pub. No.: US 2006/0293510 A1
(43) Pub. Date: Dec. 28, 2006

(54) Metal complex type nucleic acid

(75) Inventors: Mitsuhiko Shionoya, Matsudo-shi (JP); Kentaro Tanaka, Tokyo (JP); Tatsuhisa Kato, Tokyo (JP)

Correspondence Address: C. IRVIN MCCLELLAND;OBLON, SPIVAK, MCCLELLAND, MAIER & NEUSTADT, P.C., 1940 DUKE STREET, ALEXANDRIA, VA, 22314, US

(73) Assignee: Japan Science and Technology Agency, Kawaguch-shi (JP)

(21) Appl. No.: 10/570,758

(22) PCT Filed: Sep. 8, 2003
(86) PCT No.: PCT/JP03/11427

§ 371 Date: Mar. 7, 2006

(51) Int. Cl. C07H 21/02; C07H 19/048; C12P 19/34; C07H 21/04

(52) U.S. Cl. 536/23.1; 435/91.2; 536/28.1

(57) ABSTRACT

The present invention relates to a double-stranded oligonucleotide derivative, which contains two oligonucleotide derivatives each containing at least one nucleotide derivative wherein a base portion of a nucleotide is substituted with a metal coordination group that is resistant to oxidation and metal atoms, wherein the double strands are formed by coordination of each metal coordination group contained in each oligonucleotide to a metal atom so as to form a complex.

### TECHNICAL FIELD

The present invention relates to a metal complex type nucleic acid comprising oligonucleotide derivatives having metal coordination groups and metal atoms, a method for producing such metal complex type nucleic acid, and one-dimensional arraying of metal atoms in such metal complex type nucleic acid.

### BACKGROUND ART

Studies to develop novel derivatives of biomolecules have been conducted worldwide. Construction of a hierarchical structure via self-assembly as observed in natural biomolecules has been recognized as an important approach in the development of a self-assembling nanostructural molecule or material. Natural biomolecules comprise limited types of component (e.g., nucleosides, amino acids, lipids, and carbohydrates). These molecules are chemically diverse and can be polymerized or assembled almost infinitely. Furthermore, the recent development of chemical synthesis and biotechnology has made possible to produce molecular constructs that have never before existed by arraying such biomolecular components.

Subsequently, the introduction of a metal complex into a biomolecule has become recognized as an important motif in the design and synthesis of a functional biopolymer. Among many types of biomolecule, DNA molecules have a variety of structures (e.g., single-stranded or double-stranded helix, triplex, hairpin structure, and cyclic structure) and have highly regulated functions. Thus, such DNA molecules have been attractive to many researchers.

DNA is a biopolymer comprising 4 types of nucleoside unit having different nucleobases. These components are bound in a specific order that reflects genetic information via phosphodiester bonds. In contrast to the complexity of genetic information, a base pairing process that takes place between complementary DNA or RNA strands is simple. Hydrogen bonding and stacking interactions between nucleobases are important factors for stabilizing complementary DNA strands. In particular, hydrogen bonding plays an important role in the specific recognition that takes place between DNA strands.

Under such circumstances, many studies to alter DNA surfaces with metal complexes have been conducted (Hurley, D. J. et al., J. Am. Chem. Soc. 1998, 120, 2194, and Rack, J. J. et al., J. Am. Chem. Soc., 2000, 122, 6287). However, almost no studies concerning alteration of the central part of DNA have been conducted. The present inventors have discovered that base pairs formed with hydrogen bonds existing in natural DNA can be substituted with alternative base pairs. The present inventors thus have succeeded in producing a metal complex type DNA by directly altering DNA bases themselves so as to produce metal coordination nucleobases and then by pairing two nucleobases via a metal-ion-coordinating structure (U.S. Pat. Nos. 6,011,143 and 6,350,863).

However, such metal complex type DNA produced as above is extremely unstable against air oxidation and the like, and is poor in terms of practical utility for arraying and integrating metal atoms. Furthermore, types of metal atom that can be incorporated are limited, and the control and arrangement of the desired number of metal atoms have also been difficult.

In the meantime, almost no methods that involve one-dimensionally arraying an arbitrary number of metal atoms using non-biological techniques have been known. There are few existing methods that involve such one-dimensional arraying. However, some of such methods require the use of very complicated synthesis methods. Furthermore, some result in poor moldability or low practical utility because the methods are based on crystallization, which limits the type and number of metal atoms.

### DISCLOSURE OF THE INVENTION

An object of the present invention is to provide a novel structure that enables one-dimensional arraying of metal atoms and can stably exist. Another object of the present invention is to control the number and the types of metal atoms to be arrayed in such structure and the spin quantum numbers.

As a result of intensive studies to achieve the above objects, the present inventors have discovered that the above objects can be achieved with a double-stranded oligonucleotide derivative (which may also be referred to as a metal complex type nucleic acid in this description) that is formed of metal atoms and oligonucleotide derivatives containing a nucleotide derivative wherein a base portion of a nucleotide is substituted with a metal coordination group that is resistant to oxidation. Thus, the present inventors have completed the present invention.

The present invention encompasses the following inventions.

(1) A double-stranded oligonucleotide derivative, which contains two oligonucleotide derivatives each containing at least one nucleotide derivative wherein a base portion of a nucleotide is substituted with a metal coordination group that is resistant to oxidation and metal atoms, wherein the double strands are formed by coordination of each metal coordination group contained in each oligonucleotide derivative to a metal atom so as to form a complex.

(2) The double-stranded oligonucleotide derivative according to (1), wherein the oligonucleotide derivatives contain natural nucleotides.

(3) The double-stranded oligonucleotide derivative according to (1), wherein the metal coordination group has a stability constant of 10²M⁻¹ or more.

(4) The double-stranded oligonucleotide derivative according to (1), wherein the metal coordination group is selected from the following groups:

a 2-, a 3-, or a 4-pyridyl group that may be substituted;

a ring group having a group selected from hydroxyl, mercapto, amino, alkoxy, thioether, and phosphine groups and an oxo or a thioxo group at vicinal position, and containing a conjugated unsaturated bond and

a saturated organic group having an amino or a mercapto group at vicinal position, and optionally having an hetero atom.

(5) The double-stranded oligonucleotide derivative according to (1), wherein the metal coordination group is selected from the following groups:

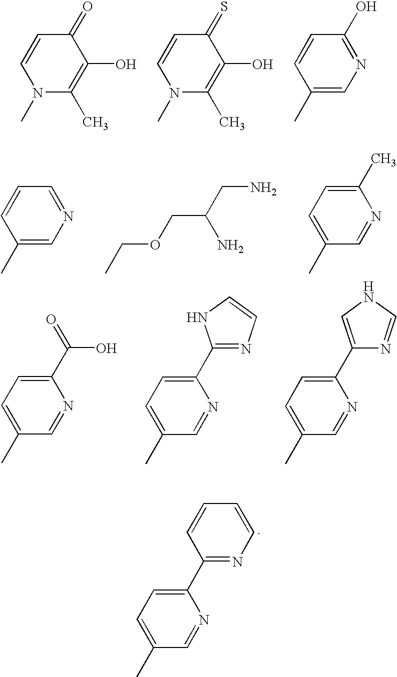

(6) The double-stranded oligonucleotide derivative according to (1), wherein the metal atoms are the same or different, and are selected from Cu²⁺, Cu⁺, Al³⁺, Ga³⁺, La³⁺, Fe³⁺, Co³⁺, As³⁺, Si⁴⁺, Ti⁴⁺, Pd²⁺, Pt²⁺, Pt⁴⁺, Ni²⁺, Ag⁺, Hg⁺, Hg²⁺, Cd²⁺, Au⁺, Au³⁺, Rh⁺, and Ir⁺.

(7) The double-stranded oligonucleotide derivative according to (1), which is not a double-stranded oligonucleotide derivative having only one metal coordination group represented by the following formula: 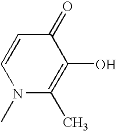
in each oligonucleotide derivative and having Cu²⁺ as a metal atom.

(8) The double-stranded oligonucleotide derivative according to (1), wherein each oligonucleotide derivative contains a plurality of nucleotide derivatives and the number of metal atoms contained is the same as the lower of the two following numbers: the number of nucleotide derivatives in one oligonucleotide derivative; and the number of nucleotide derivatives in the other oligonucleotide derivative.

(9) The double-stranded oligonucleotide derivative according to (8), wherein a plurality of nucleotide derivatives successively exist in each oligonucleotide derivative.

(10) The double-stranded oligonucleotide derivative according to (9), wherein the metal atoms have magnetism and the electron spins of a plurality of the contained metal atoms are parallel.

(11) The double-stranded oligonucleotide derivative according to (9), wherein the metal coordination groups and the metal atoms form metal complexes with a planar four-coordinate structure.

(12) A method for synthesizing a double-stranded oligonucleotide derivative that contains two oligonucleotide derivatives each containing at least one nucleotide derivative wherein a base portion of a nucleotide is substituted with a metal coordination group that is resistant to oxidation and metal atoms, wherein the double strands are formed by coordination of each metal coordination group contained in each oligonucleotide derivative to a metal atom so as to form a complex, which comprises the steps of:
synthesizing an oligonucleotide derivative by binding each other nucleotide derivatives wherein a base portion is substituted with a metal coordination group that is resistant to oxidation and optionally nucleotides by the phosphoramidite method; and
binding two oligonucleotide derivatives to each other by coordinating metal atoms to the metal coordination groups of the oligonucleotide derivatives.

(13) The synthesis method according to (12), wherein the step of synthesizing an oligonucleotide derivative is carried out such that a plurality of nucleotide derivatives are incorporated.

(14) A nucleoside derivative, which is represented by the following formula:

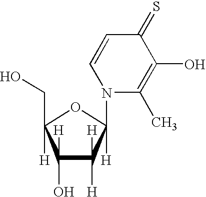

(15) A nucleoside derivative, which is represented by the following formula:

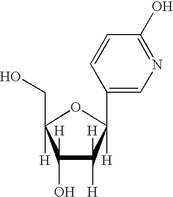

The present invention will be described in detail below.

The double-stranded oligonucleotide derivative (hereinafter may also be referred to as a metal complex type nucleic acid) of the present invention has a double-stranded structure. In such double-stranded structure, two oligonucleotide derivatives each containing at least one nucleotide derivative wherein a base portion of a nucleotide is substituted with a metal coordination group that is resistant to oxidation are bound to each other. Furthermore, each metal coordination group contained in each oligonucleotide derivative is coordinated to a metal atom, so as to form a complex. Thus, the above oligonucleotide derivatives are bound to each other to form double strands.

In the present invention, “nucleotide derivative” means a compound having a structure wherein a base portion in a nucleotide is substituted with a metal coordination group. Furthermore, “oligonucleotide derivative” means an oligonucleotide derivative having a structure wherein at least one nucleotide in an oligonucleotide is substituted with the above nucleotide derivative. An “oligonucleotide derivative” in the present invention contains at least one nucleotide derivative, may also contain a natural nucleotide, or may consist only of a nucleotide derivative. Furthermore, “metal coordination group” in the present invention means a group having a metal coordination portion capable of forming a complex by coordination to a metal atom. Specifically, such group has the functions of a ligand.

Specifically, the double-stranded oligonucleotide derivative of the present invention has a natural double helix structure comprising two oligonucleotides, wherein a base portion of at least one nucleotide in each oligonucleotide strand is substituted with a metal coordination group. When two complementary oligonucleotide derivatives form a double helix, nucleotides in the one strand existing on positions corresponding to positions where nucleotide derivatives exist in the complementary oligonucleotide derivative are also nucleotide derivatives. That is, in the double helix structure of the double-stranded oligonucleotide derivative of the present invention, metal coordination groups bound to sugar moieties of nucleotide derivatives exist facing each other. Metal coordination groups that exist at corresponding positions in each oligonucleotide derivative are coordinated together to a metal atom, thereby forming a metal complex structure. Such complex structure causes two oligonucleotide derivatives to bind to each other. Hence, the number of metal coordination groups contained in a complementary strand of an oligonucleotide derivative is generally the same as that in the other strand. In view of stabilization of such double helix structure, it is preferable that the above metal coordination groups facing each other are the same.

It is known that a natural nucleic acid has a double helix structure via complementary hydrogen bonds between base-pair-forming bases. In contrast, in the case of the metal complex type nucleic acid of the present invention, a group having a metal-coordinating site is introduced into an oligonucleotide. A double helix structure is then formed using a metal complex structure instead of using a hydrogen bond in order to apply a nucleic acid structure that originally governs genetic information to a functional material.

The double-stranded oligonucleotide derivative of the present invention is characterized by having a structure wherein a base portion of a nucleotide is substituted with a metal coordination group that is resistant to oxidation. “Metal coordination group that is resistant to oxidation” in the present invention means a metal coordination group that is not oxidized by oxygen in air or a solvent at normal temperature and under normal pressure.

Furthermore, as the metal coordination group of the present invention, a metal coordination group having a stability constant (to a metal atom) of 10²M⁻¹ or more is preferable and a metal coordination group having a stability constant of between 10⁶M⁻¹ and 10³⁰M⁻¹ is further preferable. “Stability constant” has a general meaning in the art and is a measure that shows the stability of a complex. Such stability constant is indicated as an equilibrium constant when a complex is generated from a hydrated metal atom and a ligand. When a complex [MAₙ] (an aquo-ion [M(H₂O)ₙ]^{m+} is simply denoted as M by abbreviating aqua ligands) is generated from a ligand A and a metal atom M,

in M+A [symbol] MA, MA+A [symbol] MA₂, . . . , MAₙ₋₁+A [symbol] MAₙ, each equilibrium constant is represented by K₁=[MA]/[M][A], K₂=[MA₂]/[MA][A], . . . , or Kₙ=[MAₙ]/[MAₙ₋₁][A]. “[ ]” represents each concentration. Theoretically, activity should be used. A value K obtained at this time is referred to as a thermodynamic stability constant.

Regarding a method for measuring stability constants, see Arthur E. Martell and Robert M. Smith, Critical Stability Constants Vol. 1-4, Plenum Press, New York (1974), and references cited therein.

Examples of the metal coordination group of the present invention include 2-, 3-, and 4-pyridyl groups that may be substituted. Examples of substituents include, but are not specifically limited to, hydroxyl and C1-10 alkyl groups (e.g., methyl, ethyl, and propyl groups), and the like. A pyridyl group functioning as a backbone is preferably a 3-pyridyl group among 2-, 3-, and 4-pyridyl groups. Such metal coordination group is easily coordinated in a linear two-coordinate structure. Furthermore, in the case of a carbon atom adjacent to a nitrogen atom of a pyridyl group functioning as a backbone (that is, a carbon atom at position 6 in the case of a 3-pyridyl group) may be substituted with a carboxyl group, a 2-imidazolyl group, a 4-imidazolyl group, or a 2-pyridyl group, for example. Such metal coordination group functions as a group for bidentate coordination. It is thought that when a molecule is designed so that a donor atom is positioned as the third atom from a carbon adjacent to a nitrogen atom of pyridine, the resultant will function as a bidentate ligand.

Specific examples of such metal coordination group include the following groups.

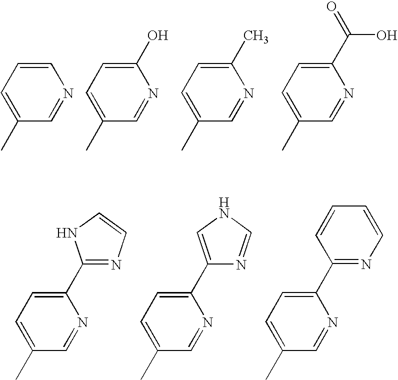

Another example of the metal coordination group of the present invention is a ring group having a group selected from hydroxyl, mercapto, amino, alkoxy, thioether, and phosphine groups, and an oxo or a thioxo group at vicinal position, and containing a conjugated unsaturated bond. “Vicinal” indicates that two substituents are each attached to adjacent carbon atoms. Furthermore, such ring group may be substituted with a substituent such as a C1-10 alkyl (e.g., a methyl, an ethyl, or a propyl group), an alkoxy, a halogen, a nitro, a cyano, an azido, or a phenyl group. Examples of the ring group are preferably 3- to 8-membered rings. More preferably, such ring group is a 5- or 6-membered ring. All members of such ring are carbon atoms, or some members of such ring are nitrogen atoms. In the case of a 6-membered ring wherein all members are carbon atoms, “ring group containing a conjugated unsaturated bond” means an aromatic ring. Preferably, a ring is a 6-membered ring that has one nitrogen atom and two double bonds and is a group that is bound to a sugar via the nitrogen atom. When a ring group is a 6-membered ring, the above two substituents preferably exist at positions 3 and 4. Such metal coordination group can be easily coordinated in a planar four-coordinate structure.

Specific examples of such metal coordination group include the following groups.

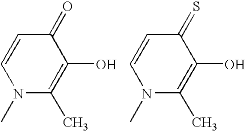

Another example of the metal coordination group of the present invention is a saturated organic group having an amino or a mercapto group at vicinal position and optionally having a hetero atom. Examples of the saturated organic group include a C3-10 and preferably a C4-5 straight or branched chain hydrocarbon group, a C5-8 and preferably a C6 cyclic hydrocarbon group, and a saturated organic group, wherein 1 to 3 carbon atoms and preferably 1 carbon atom composing a hydrocarbon group is substituted with a hetero atom (e.g., an oxygen, a nitrogen, or a sulfur atom) in the aforementioned hydrocarbon groups. A group having a hetero atom and preferably an oxygen atom is preferable. Moreover, the above saturated organic group has two vicinal substituents selected from amino and mercapto groups.

Specific examples of such metal coordination group include the following group.

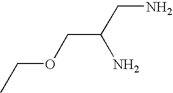

In the present invention, the following metal coordination groups are preferable in view of the stability of a double-stranded oligonucleotide derivative.

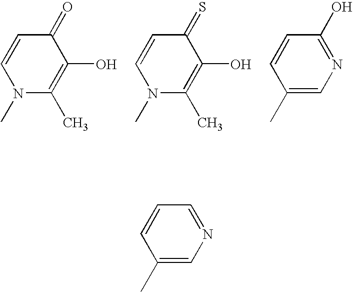

The double-stranded oligonucleotide derivative of the present invention may have a plurality of metal coordination groups of the same type or may have different metal coordination groups.

A double-stranded oligonucleotide derivative having the above metal coordination group(s) is resistant to oxidation and thus can stably exist. Hence, such double-stranded oligonucleotide derivative has practical utility as a material for one-dimensional arraying of metal atoms.

That a double-stranded oligonucleotide derivative can stably exist has the following two meanings. First, a double-stranded oligonucleotide derivative itself is not chemically changed by oxidation with oxygen in air or in a solvent, or the like. Second, the association of double strands and the association of metal atoms into double strands which are thermodynamic equilibrium reactions are sufficiently biased toward the association side. Stabilities thereof can be measured using NMR spectrum, mass spectrum, elementary analysis, absorption spectrum, electron-spin resonance spectrum, or the like.

Examples of metal atoms in the present invention include both metal atoms having no electrical charges and metal atoms having electrical charges which are namely metal ions. In the double-stranded oligonucleotide derivative of the present invention, examples of central metal atoms forming a complex with metal coordination groups are not specifically limited, as long as they can form a complex, and include, for example, Cu²⁺, Cu⁺, Al³⁺, Ga³⁺, La³⁺, Fe³⁺, Co³⁺, As³⁺, Si⁴⁺, Ti⁴⁺, Pd²⁺, Pt²⁺, Pt⁴⁺, Ni²⁺, Ag⁺, Hg⁺, Hg²⁺, Cd²⁺, Au⁺, Au³⁺, Rh⁺, and Ir⁺. In the present invention, metal atoms belonging to d-block elements and metal ions thereof are preferable. In view of coordination form, a d⁸ metal atom and a d¹⁰ metal atom are more preferable and Cu²⁺is particularly preferable. Here, “d⁸ metal atom” means metal atoms and metal ions having eight d-electrons.

A metal coordination group to be introduced into an oligonucleotide is preferably selected in accordance with the above central metal atom and a metal complex structure to be formed. For example, based on coordination number, electrical charge, and coordinate structure, a central metal atom and a metal coordination group can be selected.

In the double-stranded oligonucleotide derivative of the present invention, the desired number of metal atoms can be introduced by regulating the number of nucleotide derivatives contained in an oligonucleotide derivative. Furthermore, in each oligonucleotide, metal atoms can be successively arrayed within a double-stranded oligonucleotide derivative by successively arranging nucleotide derivatives having metal coordination groups. Generally, the same number of metal coordination groups is contained in each oligonucleotide derivative. Thus, the same number of metal atoms as that of metal coordination groups is introduced. When the numbers of metal coordination groups contained in each oligonucleotide derivative differ from each other, the number of metal atoms to be introduced into double strands is the same as the lower number of metal coordination groups. Successive arraying of metal atoms enables production of a very thin wire of metal atoms and facilitates electron transfer between metal atoms. Thus, such wire can exert excellent functions as a molecular electric wire. Such one-dimensional arraying of a plurality of metal atoms has been achieved for the first time by the present invention. Moreover, the double-stranded oligonucleotide derivative of the present invention can be used in a solution of a molecule wherein metal atoms are arrayed, therefore, is advantageous in that the derivative has high moldability and a device can be easily produced using the derivative.

Embodiments when metal atoms are successively arrayed in the double-stranded oligonucleotide derivative of the present invention are illustrated below. 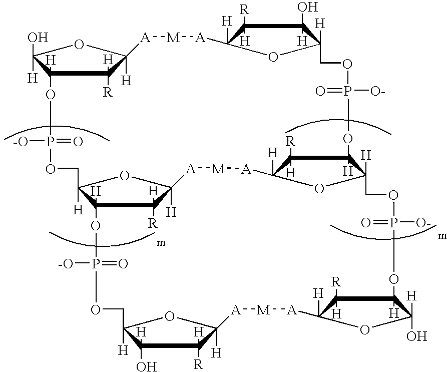
In the above formula, “A” represents the same or different metal coordination groups,

“M” represents the same or different metal atoms,

“R” represents H or OH,

“m” represents an integer between 0 and 498 and preferably an integer between 0 and 98, and

“A” and “M” form a metal complex.

When “R” is H, a metal complex type DNA is formed. When “R” is OH, a metal complex type RNA is formed.

In an embodiment wherein metal atoms are successively arrayed, a metal complex that is formed within a double-stranded oligonucleotide derivative has preferably a planar four-coordinate structure and a linear two-coordinate structure. That is because the most regular array can be accomplished by stacking of metal complexes within oligonucleotide derivative double strands.

Examples of metal atoms appropriate for the aforementioned planar four-coordinate structure include a d⁸ metal atom, specifically Rh⁺, Ir⁺, Ni²⁺, Pd²⁺, Pt²⁺, Au³⁺ ions and the like. Another example is a Cu²⁺ ion which has a large Jahn-Teller effect. Examples of metal atoms appropriate for the aforementioned linear two-coordinate structure include a d¹⁰ metal atom, specifically Cu⁺, Ag⁺, Au⁺, and Hg²⁺.

As metal coordination groups that can be used in the embodiment wherein metal atoms are successively arrayed, metal coordination groups that can form the above planar four-coordinate complex or linear two-coordinate complex with metal atoms are preferable. Furthermore, a bidentate metal coordination group, that is, a metal coordination group with which two electron-donating bonds can be formed per metal atom, and with which a total of four electron-donating bonds can be formed with metal coordination groups in two oligonucleotide derivatives, is preferable.

Examples of such metal coordination groups include the following group.

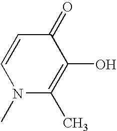

The following metal coordination group is preferable for arraying Cu²⁺.

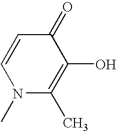

The following metal coordination group is preferable for arraying Pd²⁺, Pt²⁺, and Ni²⁺.

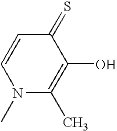

The following metal coordination group is preferable for arraying Ag⁺ and Hg²⁺.

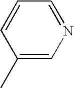

Furthermore, in the embodiment wherein metal atoms are successively arrayed, a magnetic material can be produced using metal atoms having magnetism. Surprisingly, in the double-stranded oligonucleotide derivative of the present invention, it has been revealed that when metal atoms having magnetism are successively arrayed, the electron spins of a plurality of metal atoms are oriented in parallel.

The number of metal atoms to be introduced can be regulated by regulating the number of metal coordination groups to be introduced. Thus, the spin quantum number in the double-stranded oligonucleotide derivative of the present invention can also be regulated. Accordingly, the double-stranded oligonucleotide derivative of the present invention can function as a very small magnet and is also promising as a magnetic polymer material.

Metal atoms having magnetism are not specifically limited, as long as they have unpaired electrons. Transition metal atoms having unpaired electrons are preferable. Specific examples of such metal atoms having magnetism include Sc, Ti, V, Cr, Mn, Fe, Co, Ni, Cu, Y, Zr, Nb, Mo, Ru, Rh, La, Ce, Pr, Nd, Pm, Sm, Eu, Gd, Tb, Dy, Ho, Er, Tm, Yb, Lu, Hf, Ta, W, Re, Os, Ir, and ions thereof having unpaired electrons. In particular, a Cu²⁺ ion is preferable.

The double-stranded oligonucleotide derivative of the present invention can be synthesized by the following method, for example.

Single-stranded oligonucleotide derivatives for the formation of double strands can be synthesized as follows. First, nucleoside derivatives wherein base portions of nucleosides are substituted with metal coordination groups are prepared. In addition, a method for synthesizing such nucleoside derivatives is described later.

Next, the hydroxyl group at position 5′ of a ribofuranose ring of the nucleoside derivative is dimethoxytrimethylated. The hydroxyl group at position 3′ is then changed to phosphoramidite, thereby producing a nucleotide derivative. The nucleotide derivative is then subjected to a DNA synthesizer. By the use of a phosphoramidite method that is known as a general method for synthesizing nucleic acids, oligonucleotide derivatives are synthesized. Finally, dimethoxytrityl groups and the like that are protecting groups are removed, so as to obtain single-stranded oligonucleotide derivatives for the formation of the double-stranded oligonucleotide derivative of the present invention.

The oligonucleotide derivative of the present invention may be formed with only nucleotide derivatives as described above or may also contain natural nucleotides. In the latter case, nucleotide derivatives and natural nucleotides are appropriately bound using a DNA synthesizer according to the above synthesis method.

In the case of DNA synthesis, a synthesis technique that involves aligning nucleobases in an arbitrary sequence has already been established. The hydroxyl group at position 5′ of a deoxynucleoside having a nucleobase (adenine, guanine, cytosine, or thymine) is dimethoxytritylated. The hydroxyl group at position 3′ is then phosphoramidited to obtain a deoxynucleoside derivative that is a nucleotide. The nucleotide is then placed in a commercially available automatic DNA synthesizer and then a predetermined base sequence is designated. Then, for example, a 2- to 100-base-long DNA can easily be synthesized.

The double-stranded oligonucleotide derivative of the present invention can also be synthesized by the phosphoramidite method using such DNA synthesizer and using nucleoside derivatives wherein the above base portions are substituted with metal coordination groups, and various natural nucleosides, if desired. Thus, oligonucleotide derivatives into which metal-coordinating sites have been introduced can be obtained. When such method is used, various nucleoside derivatives and nucleosides can be arrayed in an arbitrary order. Hence, metal coordination groups can be arranged at arbitrary positions in an oligonucleotide derivative. Furthermore, the length of an oligonucleotide derivative is not limited, either. Thus, a double-stranded oligonucleotide derivative having a desired length can be produced by producing oligonucleotide derivatives with the desired length. The length of the double-stranded oligonucleotide derivative of the present invention ranges from 1 to 500 bases, preferably 1 to 100 bases, and more preferably 2 to 30 bases, for example.

Thus obtained two oligonucleotide derivatives complementary to each other form the double-stranded oligonucleotide derivative of the present invention as a result of coordination of metal coordination groups of each oligonucleotide derivative to metal atoms to form a double-stranded structure.

Metal complex formation, that is, the incorporation of metal atoms into double strands, can be carried out by causing two oligonucleotide derivatives that have metal coordination groups at corresponding positions and that are complementary to each other to coexist with metal atoms in a solvent. Metal atoms can be provided by adding a salt that donates a desired metal atom into a solvent. A solvent to be used herein is not specifically limited. For example, an aqueous solution can be used. When an aqueous solution is used, pH region is preferably selected such that a ligand has higher biding affinity to a target metal atom than that of a proton as a Lewis acid, and that a metal atom has higher biding affinity to a ligand than that of a hydroxium ion as a Lewis base. Moreover, low temperatures are desired, as long as a solvent is not frozen and a solute is not precipitated.

In the absence of metal atoms, oligonucleotide derivatives having nucleotide derivatives wherein bases are substituted with metal coordination groups are hardly associated with each other and the stability of the resultant double strands is low. By causing coexistence with metal atoms, stable double strands are formed. Accordingly, formation of double-stranded oligonucleotide derivative can be controlled depending on the presence or absence of and concentration of metal atoms.

The present invention also relates to a nucleoside derivative wherein a base portion of a nucleoside is substituted with a metal coordination group.

Examples of the nucleoside derivative of the present invention include the following derivatives.

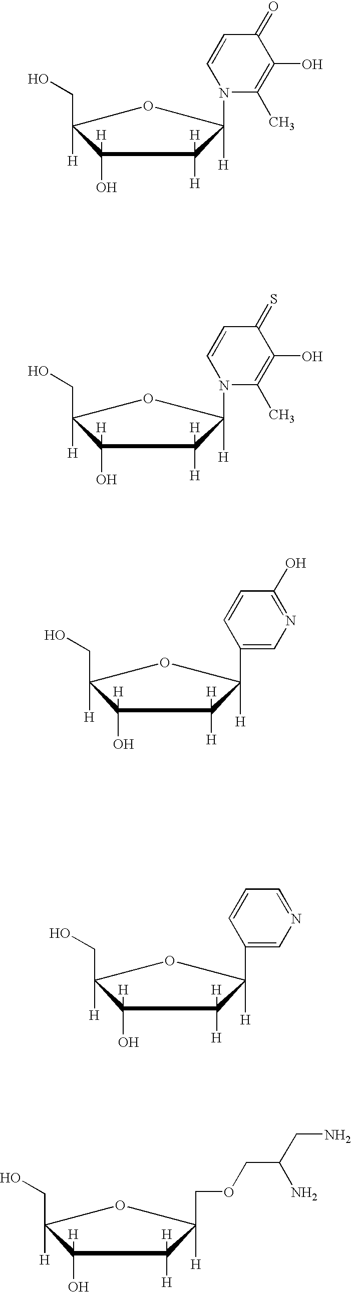

The nucleoside derivative of the present invention is generally obtained by obtaining the backbone structure of a nucleoside by condensation of deoxyribose derivatives and metal ligand sites using a Friedel-Crafts reaction, condensation of deoxyribonolactone derivatives and lithiated metal ligand sites, or addition reaction of glycal with organic-metallized metal ligands, followed by a deprotecting reaction.

As described above in the present invention, metal atoms can be introduced at arbitrary positions in a double-stranded oligonucleotide derivative. For example, a single metal atom can also be introduced or metal atoms can also be successively introduced. For example, an oligonucleotide derivative with metal coordination groups at arbitrary positions can be obtained using an automatic DNA synthesizer. Specifically, an artificial nucleic acid is designed based on functions to be conferred and then coordinating sites and metal atoms are selected. Thus, a compound having a structure wherein arbitrary metal atoms are arranged at arbitrary positions can easily be synthesized.

### BRIEF DESCRIPTION OF THE DRAWINGS

FIG. 1 shows, as an embodiment of the present invention, a metal complex type DNA structure wherein Cu²⁺ ions are successively arranged in two strands of oligonucleotide derivatives each having hydroxypyridone groups.

FIG. 2 shows the results of measuring UV absorption spectra with varying molar ratio of Cu²⁺ ions to oligonucleotide double strands, in the presence of complementary oligonucleotide derivative strands each having 5 hydroxypyridone groups.

FIG. 3 shows the results of measuring changes in UV absorption at 307 nm against the molar ratio of Cu²⁺ ions to oligonucleotide double strands, wherein measurement was carried out for every number of hydroxypyridone groups (n) contained in each oligonucleotide.

FIG. 4 shows the results of measuring the CD spectra of metal complex type DNAs containing 1 to 5 Cu²⁺ ions.

FIG. 5 shows the results of measuring the CW-EPR spectra of metal complex type DNAs containing 1 to 5 Cu²⁺ ions using an X-band spectrometer.

### BEST MODE OF CARRYING OUT THE INVENTION

The present invention will be further described in detail by referring to the following examples. However, the present invention is not limited by these examples.

### EXAMPLE 1

### Synthesis of a Nucleoside Derivative and a Nucleotide Derivative Having Structures Wherein Nucleobases are Substituted With Metal Coordination Groups

A nucleoside derivative and a nucleotide derivative having hydroxypyridone groups were synthesized according to the following scheme. 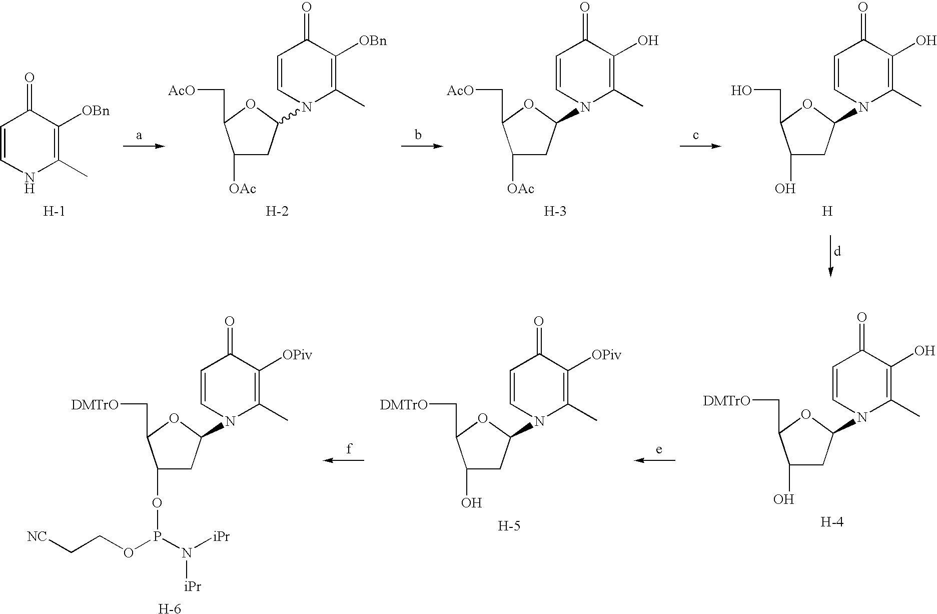
In the above scheme, “Bn” represents benzyl, “Piv” represents pivaloyl, and “DMTr” represents 4,4′-dimethoxytrityl.

1,3,5-tri-O-acetyl-2-deoxy-D-ribofuranose and 2-methyl-3-(benzyloxy)-4-pyridone were synthesized according to the methods of Gold, A. et al., (Nucleocides Nucleotides 1990, 9, 907) and Harris, R. L. N. et al., (Aust. J. Chem. 1976, 29, 1329). Next, 2-methyl-3-(benzyloxy)-4-pyridone (504 mg and 2.34 mmol) and a catalytic amount of ammonium sulfate were dissolved in hexamethyldisilazane (5 mL of HMDS). The reaction mixture was heated for 2 hours under reflux and then an excessive amount of HMDS was distilled off. A CH₃CN (25 mL) solution of 1,3,5-tri-O-acetyl-2-deoxy-D-ribofuranose (669 mg and 2.57 mmol) was added to the thus obtained residue. Subsequently, trimethylsilyltrifluoromethanesulfonate (465 μl and 2.57 mmol) was added dropwise to the reaction mixture. The obtained solution was stirred at room temperature for 24 hours. The reaction was stopped with a saturated sodium hydrogencarbonate aqueous solution, and then the solvent was distilled off. The residue was dissolved in CH₂Cl₂. After the organic phase was washed with a saturated NaHCO₃ aqueous solution and water, the resultant was dried with anhydrous Na₂SO₄. After the solvent was distilled off, the residue was purified by silica gel column chromatography (CHCl₃—CH₃OH (100:1)). Thus, a compound H-2 wherein the ratio of α-anomer to β-anomer was 3:7 was obtained.

The compound H-2 (3.7 g and 8.9 mmol) was dissolved in AcOEt (100 mL) and then 10% Pd/C (500 mg and 0.47 mmol) was added to the reaction mixture. The suspension was stirred heavily under H₂ atmosphere for 2 hours. After the completion of the reaction, Pd/C was filtered off, the solvent was distilled off, and then the residue was recrystallized from EtOH. Thus, a desired compound H-3 was obtained (870 mg and 30%).

A 28% NH₄OH aqueous solution (10 ml) was added to a methanol (40 mL) solution of the compound H-3 (998 mg and 3.07 mmol). The mixture was stirred at room temperature for 3 hours, the solvent was distilled off, and then the residue was solidified in AcOEt. Thus a compound H was obtained as a colorless solid substance. Mp: 141.0° C. to 143.0° C.

DMTr-Cl (570 mg and 1.68 mmol) was added to an anhydrous pyridine (2 ml) solution of the compound H (290 mg and 1.20 mmol). The reaction mixture was stirred at room temperature for 2 hours. After the reaction was stopped with MeOH, the mixture was poured into ice water (100 ml), followed by extraction with CH₃Cl. The organic phase was dried with anhydrous MgSO₄ and then condensed. The residue was purified by silica gel column chromatography (CHCl₃—CH₃OH (100:1)). Thus, a compound H-4 (498 mg and 77%) was obtained.

Pivalic anhydride (403 μL and 2.12 mmol) was added to a THF (7.7 mL) solution of the compound H-4 (1.05 g and 1.93 mmol) and iPr₂EtN (404 μL and 2.32 mmol). The solution was stirred at room temperature for 15 hours. The reaction mixture was poured into CHCl₃ (150 ml) and then washed with a saline solution. The organic phase was dried with MgSO₄ and then the solvent was distilled off. The residue was purified by silica gel column chromatography (CHCl₃) and then by alumina column chromatography (CHCl₃). Thus, a compound H-5 (741 mg and 61%) was obtained.

2-cyanoethyl N,N-diisopropylchlorophosphoramidite (267 μl and 1.20 mmol) was added to a CHCl₃ (10 mL) solution of the compound H-5 (342 mg and 545 μmol) and N,N-diisopropylethylamine (238 μl and 1.36 mmol). 30 minutes later, the reaction mixture was poured into ice water (30 ml), followed by extraction with CH₂Cl₂ (100 ml). The organic phase was washed with water and then dried with MgSO₄. The solvent was distilled off and then the residue was purified by silica gel column chromatography. Thus, the diastereo mixture of a compound H-6 was obtained (275 mg and 61%).

### EXAMPLE 2

### Synthesis of a Nucleoside Derivative and a Nucleotide Derivative Having Structures Wherein Nucleobases are Substituted With Metal Coordination Groups

A nucleoside derivative and a nucleotide derivative having pyridine groups were synthesized according to the following scheme. 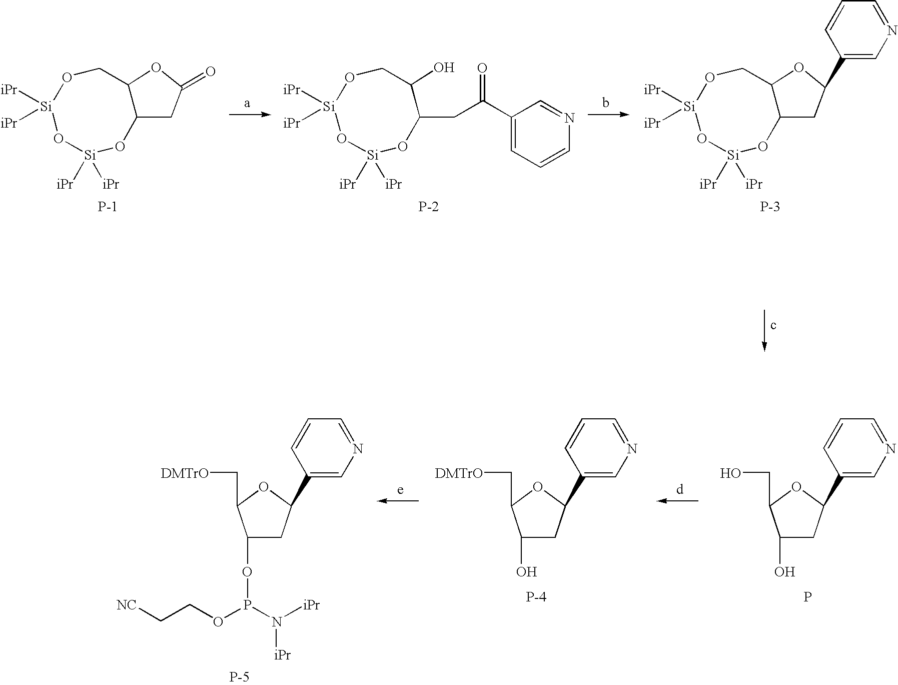
In the above scheme, “DMTr” represents 4,4′-dimethoxytrityl.

2-deoxy-3,5-O-(1,1,3,3-tetraisopropyldisiloxane-1,3-diyl)-D-ribono-1,4-lactone was synthesized according to the method of Markiewicz, W. T. (J. Chem. Res, Synop. 1979, 24). Next, a hexane solution of n-butyl lithium (1.56 M, 19.5 mL, and 30.4 mmol) was gently added to an anhydrous diethylether (180 mL) solution of 3-bromopyridine (2.75 mL and 28.5 mmol) cooled to −78° C. The thus obtained yellow solution was stirred at −78° C. for 30 minutes. 2-deoxy-3,5-O-(1,1,3,3-tetraisopropyldisiloxane-1,3-diyl)-D-ribono-1,4-lactone (10.7 g and 28.6 mmol) dissolved in anhydrous diethylether (20 mL) was added dropwise to the solution at −78° C. for 10 minutes. After 2 hours of stirring at −78° C., a saturated ammonium chloride aqueous solution (50 mL) was added to the reaction solution, so as to stop the reaction. The thus obtained mixture was extracted with diethylether (100 mL×3). The organic phase was washed with a saturated saline solution (200 mL) and then dried with anhydrous magnesium sulfate. The solvent was distilled off and then the residue was purified by silica gel column chromatography (hexane-diethylether (1:6)). Thus, a compound P-2 was obtained (7.6 g and 59%).

The compound P-2 (16.2 g and 35.7 mmol) was dissolved in CH₂Cl₂ (120 mL), and then to which triethylsilane (29.0 ml and 181 mmol) was then added at −78° C. The solution was stirred at −78° C. for 10 minutes and then a boron trifluoride diethylether complex (22.6 mL and 178 mmol) dissolved in CH₂Cl₂ (160 mL) was added dropwise over 10 minutes. The temperature of the reaction solution was elevated to −50° C., followed by 40 hours of stirring. 50 mL of a saturated ammonium chloride aqueous solution was added to stop the reaction. The mixture was extracted with diethylether (100 mL×3). The organic phase was washed with a saturated saline solution (200 mL) and then dried with anhydrous magnesium sulfate. The solvent was distilled off and then the residue was purified by silica gel column chromatography (hexane-ethyl acetate (5:1)). Thus, a β-form compound P-3 was obtained as colorless oil (2.7 g and 18%).

The compound P-3 (2.7 g and 6.2 mmol) was dissolved in tetrahydrofuran (100 mL). A tetrahydrofuran solution of tetrabutylammonium fluoride (1.0 M, 18.6 mL, and 186 mmol) was added to the solution at room temperature. The thus obtained reaction solution was stirred for 70 minutes. A saturated ammonium chloride aqueous solution (100 mL) was added to the reaction solution, so as to stop the reaction. The solution was condensed. The residue was dispersed in ethyl acetate, insoluble salt was filtered off, and then the solvent was distilled off. The thus obtained residue was purified by silica gel column chromatography (ethyl acetate). Thus, a compound P was obtained as colorless oil (1.1 g and 89%).

The compound P (141 mg and 0.72 mmol) was dissolved in anhydrous pyridine (4 mL), into which DMTr-Cl (253 mg and 0.72 mmol) was then added at room temperature. The solution was stirred at room temperature for 2.5 hours and then 20 mL of methanol was added to stop the reaction. The solvent was distilled off. 10 mL of ethanol was added to the residue, and caused azeotropy. The step was repeated twice, thereby completely removing pyridine. The residue was purified by silica gel column chromatography (ethyl acetate). Thus, a compound P-4 was obtained in a colorless form (274 mg and 76%).

The compound P-4 (577 mg and 1.16 mmol) was dissolved in CH₂CH₂ (11 mL), to which N,N-diisopropylethylamine (0.80 mL and 4.60 mmol) and 2-cyanoethyl N,N-diisopropylchlorophosphoramidite (0.54 mL and 2.42 mmol) were then added at room temperature, followed by 3 hours of stirring. 10 mL of methanol was added to stop the reaction. The solution was further stirred for 10 minutes. The solvent was distilled off. The residue was dissolved in ethyl acetate (100 mL). The solution was washed with a saturated sodium hydrogencarbonate aqueous solution (100 mL), water (100 mL×2), and a saturated saline solution (100 mL). Drying was carried out with anhydrous sodium sulfate and then the solvent was distilled off. The residue was purified by silica gel column chromatography (hexane-ethyl acetate (1:1)). Thus, a compound P-5 was obtained as colorless oil (633 mg and 80%).

### EXAMPLE 3

### Synthesis of a Nucleoside Derivative and a Nucleotide Derivative Having Structures Wherein Nucleobases are Substituted With Metal Coordination Groups

A nucleoside derivative and a nucleotide derivative having hydroxypyridine thion groups were synthesized according to the following scheme.

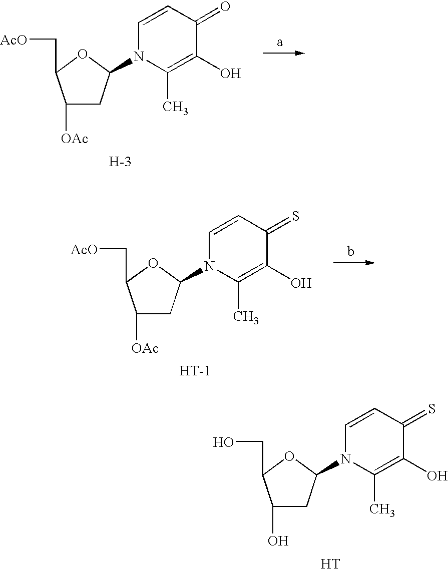

The compound H-3 (0.505 g and 1.55 mmol) and diphosphorus pentasulfide (0.362 g and 1.63 mmol) were dispersed in 7 mL of acetonitrile. N,N-diisopropylethylamine (1.1 mL and 6.16 mmol) diluted with 6.2 mL of acetonitrile was added dropwise to the solution during cooling with ice and stirring. The reaction solution was directly stirred for 4 hours and then poured into cold water, followed by extraction with methylene chloride. The organic phase was washed with water and then dried with anhydrous magnesium sulfate. The solvent was distilled off and then the residue was recrystallized from isopropanol. Thus, a compound HT-1 was obtained in a yellow crystalline form (0.351 g and 61%).

The compound HT-1 (0.448 g and 1.31 mmol) was dissolved in 20 mL of methanol to which 5 mL of concentrated ammonia water was then added, followed by 4 hours of stirring. The solvent was distilled off. By the addition of ethyl acetate to the thus obtained residue, a compound HT was obtained as a precipitate (0.278 g and 82%).

### EXAMPLE 4

### Synthesis of Oligonucleotide Derivatives

Five oligonucleotide derivatives containing 1 to 5 hydroxypyridone groups, respectively, were synthesized. Synthesis was carried out based on standard β-cyanoethylphosphoramidite chemistry using an ABI 394 DNA synthesizer (PE Biosystems). The produced oligonucleotide derivatives are as shown below.

| | |
|---|---|
| d (5′-GHC-3′) | (SEQ ID NO: 1) |
| d (5′-GHHC-3′) | (SEQ ID NO: 2) |
| d (5′-GHHHC-3′) | (SEQ ID NO: 3) |
| d (5′-GHHHHC-3′) | (SEQ ID NO: 4) |
| d (5′-GHHHHHC-3′) | (SEQ ID NO: 5) |

Here, “H” means the nucleotide derivative having hydroxypyridone groups produced in the above Example 1. The oligonucleotide derivatives represented by the above SEQ ID NOS: 1 to 5 are self-complementary strands. Thus, the same sequences can form a double-stranded oligonucleotide derivative.

Reagents, concentrations, and the like used herein were similar to those used in the synthesis of natural DNA oligomers. Synthesis was carried out at a 1-μmol scale according to the manufacturer's protocols. The sole change added to a general synthesis cycle was extension of the coupling time to 15 minutes. Oligomers were removed from supports and then treated with 25% NH₃ (55° C. and 12 hours), so as to carry out deprotection. Crude oligonucleotide derivatives were purified and then detritylated.

### EXAMPLE 5

### UV Absorption Measurement

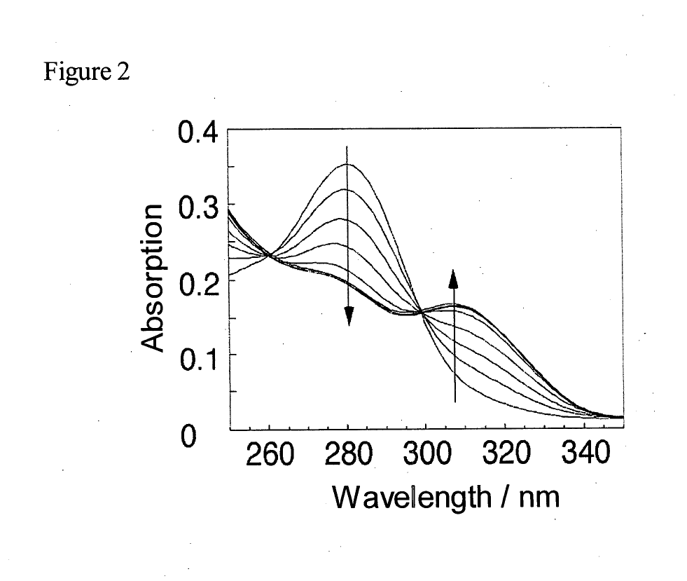

In the presence of the complementary strands (SEQ ID NO: 5) of oligonucleotide derivatives each having 5 hydroxypyridone groups, UV absorption spectra were measured (Hitachi U-3500 spectrometer) with varying molar ratio of Cu²⁺ ions to oligonucleotide derivative double strands (double strands of oligonucleotide derivatives not containing metal atoms). FIG. 2 shows the results. When the amount of Cu²⁺ ions was increased, absorption at 280 mn decreased and a new peak appeared in the vicinity of 307 nm. This indicates that because of the formation of Cu²⁺ complexes, deprotonation of the hydroxyl groups of hydroxypyridone groups took place.

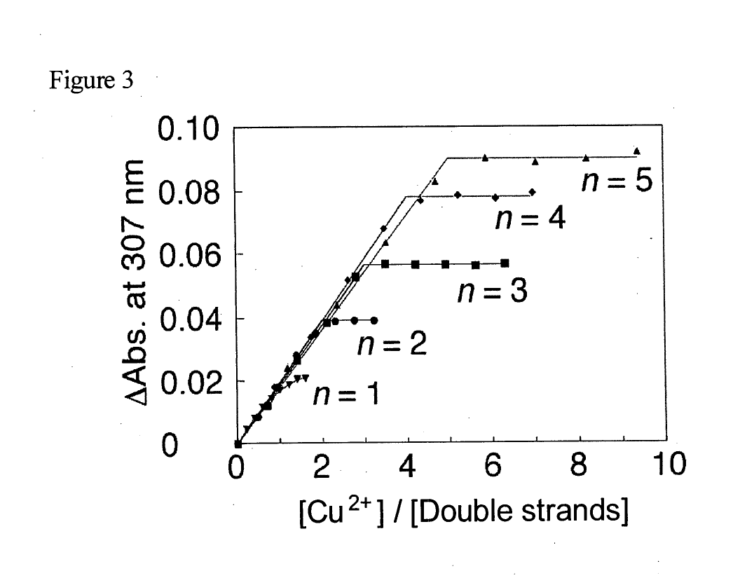

Furthermore, FIG. 3 shows changes in UV absorption at 307 nm against the molar ratio of Cu²⁺ ions to oligonucleotide derivative double strands, which were measured for every number of hydroxypyridone groups (n) contained in each oligonucleotide derivative. In the case of oligonucleotide derivative double strands with n=2, it was shown that absorption increased till [Cu²⁺]/[double strands]=2 but no increase was observed in absorption when the amount of Cu²⁺ ions was increased beyond such level. Similarly, in the case of oligonucleotide derivative double strands with n=5, absorption increased till [Cu²⁺]/[double strands]=5, but no increase was observed in absorption when the amount of Cu²⁺ ions was increased beyond such level. Here, “double strands” means the concentration of oligonucleotide derivative double strands; that is, ½ of the entire concentration of oligonucleotide derivative single strand. Based on the above results, it was shown that Cu²⁺ ions in the same number as that of hydroxypyridone groups existing in each oligonucleotide strand (n) were incorporated into the oligonucleotide derivative double strands.

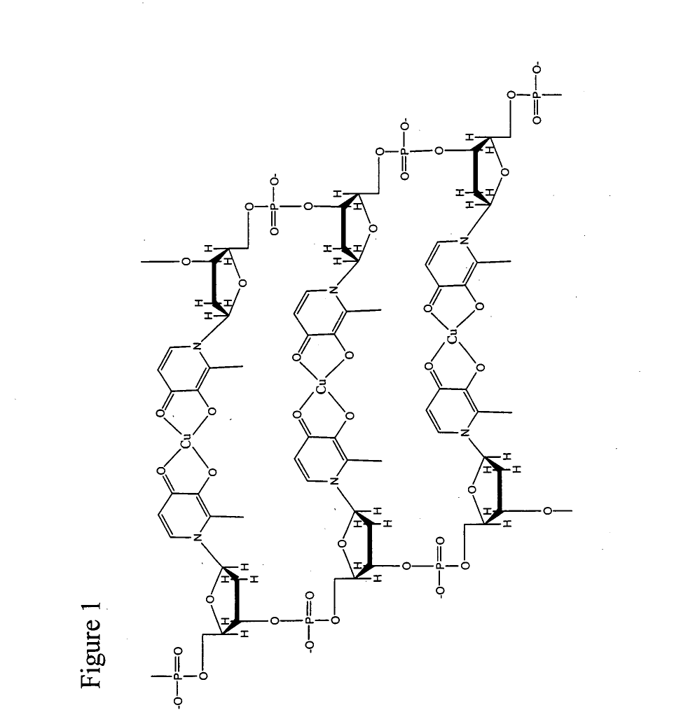

FIG. 1 shows as an embodiment of the present invention a metal complex type DNA structure that was formed by successively arranging Cu²⁺ ions in oligonucleotide derivative double strands having hydroxypyridone groups.

### EXAMPLE 6

### CD Spectrum Measurement

By the use of oligonucleotide derivatives represented by SEQ ID NOS: 3 to 12, double-stranded oligonucleotide derivatives containing Cu²⁺ ions in the same number as that of hydroxypyridone groups contained in each oligonucleotide derivative (n) were produced.

First, copper sulfate was added to a double-stranded DNA derivative (2.0 μM) dissolved in a 10 mM HEPES buffer (pH 7.0) supplemented with 50 mM NaCl, so as to result in the same concentration as that of metal complex base pairs at 25° C.

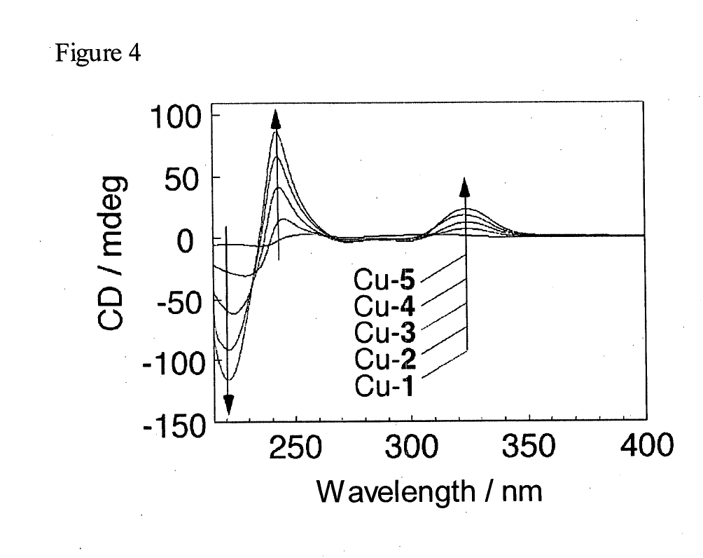

A double-stranded oligonucleotide derivative having “n (number of)” hydroxypyridone groups in each oligonucleotide derivative and containing “n (number of)” Cu²⁺ ions is denoted as a metal complex type DNA (Cu-n). For 5 types of metal complex type DNAs (Cu-1 to Cu-5), CD (circular dichroism) spectra were measured. Each metal complex type DNA (8.0 μM) was dissolved in a solution of 10 mM Hepes (pH7.0) and 50 mM NaCl and then scanned over 400−215 nm at 25° C. FIG. 4 shows the results. The spectra showed the typical characteristics of right-handed double helix DNA. Moreover, signals at approximately 324 nm indicate that deprotonation of hydroxypyridone groups took place due to the formation of complexes with Cu²⁺.

### EXAMPLE 7

### CW-EPR Spectrum Measurement

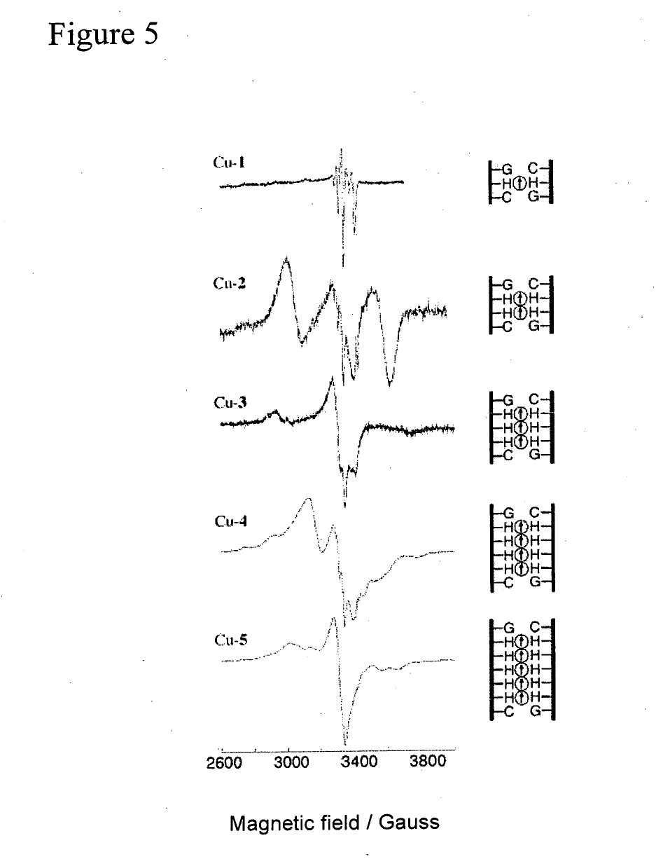

The CW-EPR (continuous wave electron paramagnetic resonance) spectra of 5 types of metal complex type DNAs (Cu-1 to Cu-5) that were the same as those in Example 6 were measured using an X-band spectrometer with a frequency of 9.4 GHz at 1.5 K. FIG. 5 shows the results.

The metal complex type DNA (Cu-1) containing one Cu²⁺ ion showed the doublet (S=½) of Cu²⁺ at the center of a planar four-coordinate field. In contrast, the metal complex type DNAs (Cu-2 and Cu-4) containing the odd numbers of Cu²⁺ ions showed convex spectra having strong central signals. On the other hand, the metal complex type DNAs (Cu-3 and Cu-5) containing the even numbers of Cu²⁺ ions showed concave spectra. The spectrum in the case of Cu-2 showed the spin state, S=1, that in the case of Cu-3 showed S=3/2, that in the case of Cu-4 showed S=2, and that in the case of Cu-5 showed S=5/2. The CW-EPR spectrum in the case of Cu-2 showed a pattern agreeing with the dipole-dipole interaction between electron spins over the entire distance between base-pair-forming bases (3.3 Å to 3.4 Å) in a natural DNA. The distance between Cu²⁺ and Cu²⁺ was estimated to be 3.7±0.1 Å.

As described above, it was shown that adjacent Cu²⁺ electron spins are aligned in parallel to generate strong magnetism by the accumulation of Cu²⁺ ions.

All publications, patents, and patent applications cited herein are incorporated herein by reference in their entirety.

### INDUSTRIAL APPLICABILITY

According to the present invention, a metal complex type nucleic acid that can stably exist can be constructed and various metal atoms can be one-dimensionally arrayed. Furthermore, the number and the types of metal atoms to be arrayed and the spin quantum numbers of the arrayed metal atoms can be controlled. Such metal complex type nucleic acid can be used for molecular electric wire or a magnetic polymer material.

1. A double-stranded oligonucleotide derivative, which contains two oligonucleotide derivatives each containing at least one nucleotide derivative wherein a base portion of a nucleotide is substituted with a metal coordination group that is resistant to oxidation and metal atoms, wherein the double strands are formed by coordination of each metal coordination group contained in each oligonucleotide derivative to a metal atom so as to form a complex.

2. The double-stranded oligonucleotide derivative according to claim 1, wherein the oligonucleotide derivatives contain natural nucleotides.

3. The double-stranded oligonucleotide derivative according to claim 1, wherein the metal coordination group has a stability constant of 10²M⁻¹ or more.

4. The double-stranded oligonucleotide derivative according to claim 1, wherein the metal coordination group is selected from the following groups:
a 2-, a 3-, or a 4-pyridyl group that may be substituted;
a ring group having a group selected from hydroxyl, mercapto, amino, alkoxy, thioether, and phosphine groups and an oxo or a thioxo group at vicinal position and containing a conjugated unsaturated bond and
a saturated organic group having an amino or a mercapto group at vicinal position and optionally having an hetero atom.

5. The double-stranded oligonucleotide derivative according to claim 1, wherein the metal coordination group is selected from the following groups:

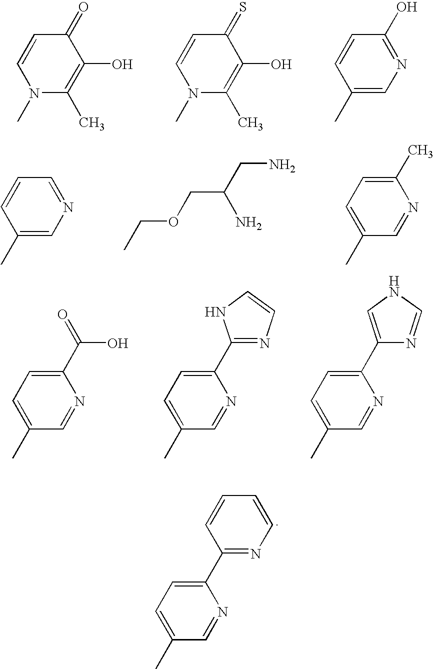

6. The double-stranded oligonucleotide derivative according to claim 1, wherein the metal atoms are the same or different, and are selected from Cu²⁺, Cu⁺, Al³⁺, Ga³⁺, La³⁺, Fe³⁺, Co³⁺, As³⁺, Si⁴⁺, Ti⁴⁺, Pd²⁺, Pt²⁺, Pt⁴⁺, Ni²⁺, Ag⁺, Hg⁺, Hg²⁺, Cd²⁺, Au⁺, Au³⁺, Rh⁺, and Ir⁺.

7. The double-stranded oligonucleotide derivative according to claim 1, which is not a double-stranded oligonucleotide derivative having only one metal coordination group represented by the following formula: 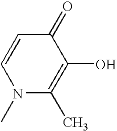
in each oligonucleotide derivative and having Cu²⁺ as a metal atom.

8. The double-stranded oligonucleotide derivative according to claim 1, wherein each oligonucleotide derivative contains a plurality of nucleotide derivatives and the number of metal atoms contained is the same as the lower of the two following numbers: the number of nucleotide derivatives in one oligonucleotide derivative; and the number of nucleotide derivatives in the other oligonucleotide derivative.

9. The double-stranded oligonucleotide derivative according to claim 8, wherein a plurality of nucleotide derivatives successively exist in each oligonucleotide derivative.

10. The double-stranded oligonucleotide derivative according to claim 9, wherein the metal atoms have magnetism and the electron spins of a plurality of the contained metal atoms are parallel.

11. The double-stranded oligonucleotide derivative according to claim 9, wherein the metal coordination groups and the metal atoms form metal complexes with a planar four-coordinate structure.

12. A method for synthesizing a double-stranded oligonucleotide derivative that contains two oligonucleotide derivatives each containing at least one nucleotide derivative wherein a base portion of a nucleotide is substituted with a metal coordination group that is resistant to oxidation and metal atoms, wherein the double strands are formed by coordination of each metal coordination group contained in each oligonucleotide derivative to a metal atom so as to form a complex, which comprises the steps of: synthesizing an oligonucleotide derivative by binding each other nucleotide derivatives wherein a base portion is substituted with a metal coordination group that is resistant to oxidation, and optionally nucleotides by the phosphoramidite method; and binding two oligonucleotide derivatives to each other by coordinating metal atoms to the metal coordination groups of the oligonucleotide derivatives.

13. The synthesis method according to claim 12, wherein the step of synthesizing an oligonucleotide derivative is carried out such that a plurality of nucleotide derivatives are incorporated.

14. A nucleoside derivative, which is represented by the following formula:

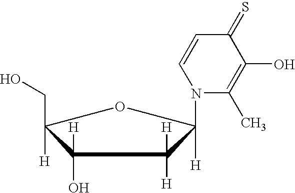

15. A nucleoside derivative, which is represented by the following formula: [chemical structure]
